(19) United States
(12) Patent Application Publication
(10) Pub. No.: US 2024/0238066 A1
(43) Pub. Date: Jul. 18, 2024

(54) Localization Apparatus

(71) Applicant: Martin A. Urban, Belvidere, IL (US)

(72) Inventor: Martin A. Urban, Belvidere, IL (US)

(21) Appl. No.: 18/154,562

(22) Filed: Jan. 13, 2023

(51) Int. Cl. A61B 90/00

(52) U.S. Cl. CPC A61B 90/39; A61B 2090/3908; A61B 2090/3983; A61B 2090/3995

(57) ABSTRACT

A localization apparatus including a hollow localization wire and an internal wire. The hollow localization wire has a securing feature configured to secure the hollow localization wire in place relative to target tissue. The internal wire is disposed in the hollow localization wire. A distal end of the internal wire protruding from the hollow localization wire is configured to emit a signal permitting the target tissue to be located.

### CROSS-REFERENCE TO RELATED APPLICATIONS

None.

### TECHNICAL FIELD

The present disclosure is generally related to the field of medical devices and, in particular, to medical devices for locating target tissue in a body of a patient.

### BACKGROUND

Before a biopsy or surgical procedure to remove a lesion within a breast (e.g., a lumpectomy procedure) or other tissue, the location of the lesion must be identified. To do so, mammography or ultrasound imaging may be used to identify and/or determine the location of the lesion before the procedure. The resulting images may be used to guide the surgeon during dissection to access and/or remove the lesion. However, such images are generally two-dimensional and therefore provide only limited guidance for localization of the lesion since the breast and any lesion to be removed are three-dimensional structures. Further, such images may provide only limited guidance in determining a proper margin around the lesion, e.g., defining a desired specimen volume to be removed.

### SUMMARY

The disclosed aspects/embodiments provide a localization apparatus. The localization apparatus provides tactile feedback to a surgeon, remains securely in place once positioned, and allows the target tissue to be precisely located and effectively removed. In addition, the localization apparatus is inexpensive and easily inserted into tissue.

These and other features will be more clearly understood from the following detailed description taken in conjunction with the accompanying drawings and claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIGS. 1A-1B illustrate a wire marker device configured to perform a localization procedure.

FIGS. 2A-2B illustrate a seed implantation device configured to perform a localization procedure.

FIGS. 3A-3B illustrate a localization apparatus according to an embodiment of the disclosure.

FIG. 4 illustrates a localization apparatus according to an embodiment of the present disclosure.

FIG. 5 illustrates a localization apparatus according to an embodiment of the present disclosure.

FIG. 6 illustrates a localization apparatus according to an embodiment of the present disclosure.

FIG. 7 illustrates a localization apparatus according to an embodiment of the present disclosure.

FIG. 8 is a method of locating target tissue in a body of a patient according to an embodiment of the present disclosure.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

To facilitate localization of a lesion (or other target tissue), a wire may be inserted into the breast immediately before a procedure. The wire may be inserted via a needle such that a tip of the wire is positioned at or near the location of the lesion. Once positioned, the wire may be secured in place using a bandage or tape applied to the patient's skin where the wire emerges from the breast. With the wire placed and secured in position, the patient may proceed to surgery to have a biopsy or lumpectomy performed.

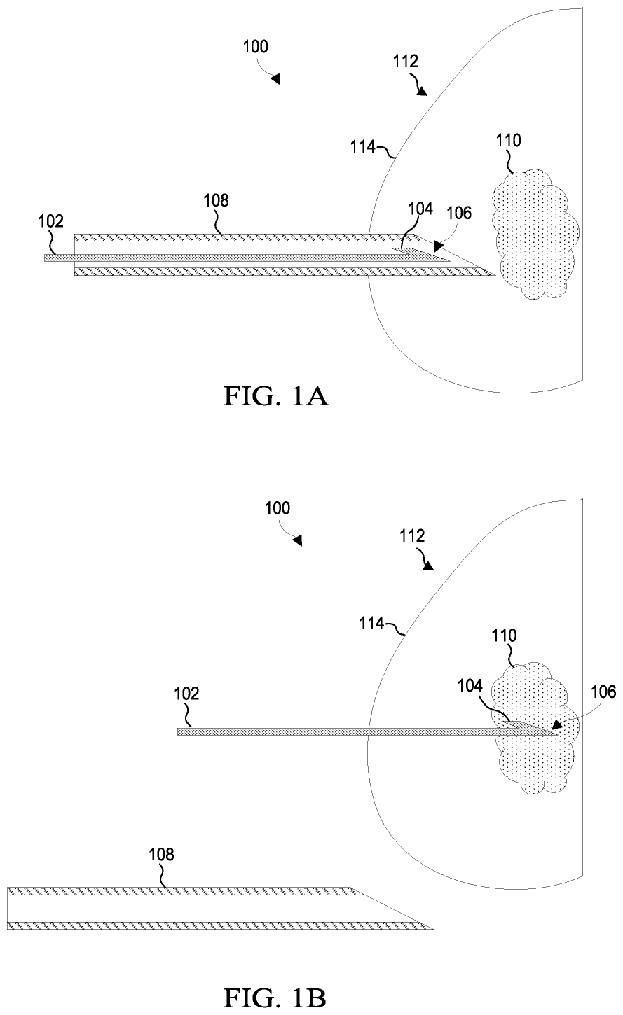

FIGS. 1A and 1B depict a wire marker device 100 configured to perform the procedure described above. As shown, the wire marker device 100 includes a semi-rigid wire 102 with a hook 104 at or near the distal end 106. During use, the semi-rigid wire 102 is forced through a needle 108 positioned near target tissue 110 of a breast 112 until the hook 104 emerges, releasing the hook 104 in the target tissue 110. Thereafter, the needle 108 is removed, which leaves the semi-rigid wire 102 protruding from the skin 114. The surgeon then makes an incision where the hook enters the skin 114 and dissects along the wire 102 down to the target tissue 110. Thus, the wire 102 and hook 104 enable a surgeon to quickly locate and remove the target tissue 110.

One problem with using a wire for localization is that the wire may move between the time of placement and the surgical procedure. For example, when the wire is not secured sufficiently, the wire may move relative to the tract used to access the lesion. Consequently, the tip of the wire may misrepresent the location of the lesion. When this occurs, the lesion may not be fully removed and/or healthy tissue may be unnecessarily removed by the surgeon performing the procedure. The surgeon may want to enter the breast at a different location than the insertion site on the localization wire, which may make it difficult to locate the lesion/wire. Thus, use of the wire alone for localization has drawbacks.

In order to overcome some of the disadvantages of using only the wire for localization, a seed (e.g., a radioactive seed) may be used for localization. For example, a needle may be introduced through a breast and into a lesion. Thereafter, the seed is ejected from the needle and into the lesion. The needle is then withdrawn, and the position of the seed is confirmed using mammography or another location technique. During a subsequent surgical procedure to remove the lesion, a hand-held probe is placed over the breast to identify the location of the seed within (or relative to) the lesion. An incision is then made and the probe is used to guide excision of the seed and the lesion.

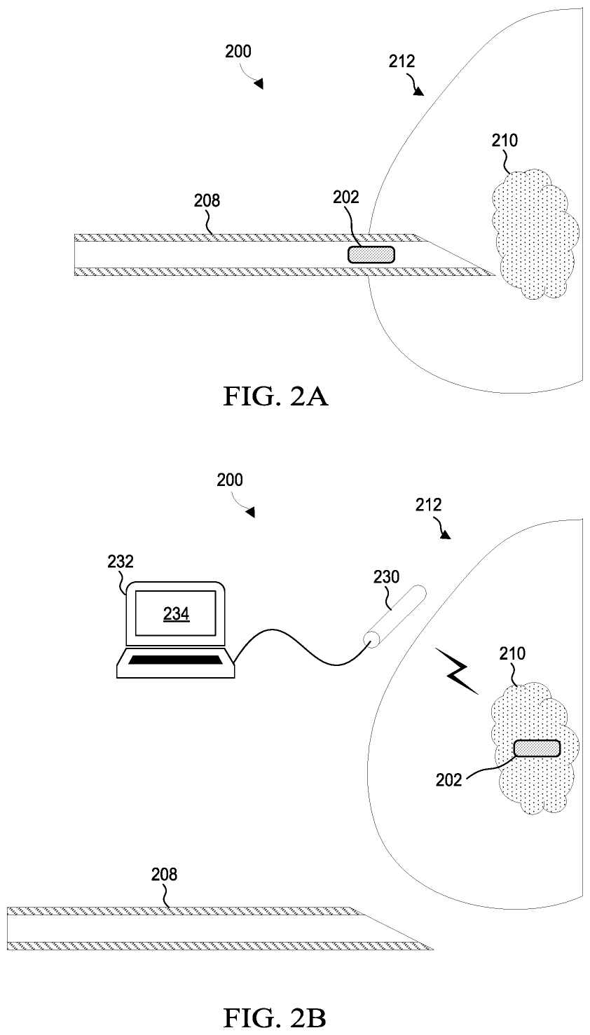

FIGS. 2A and 2B illustrate a seed implantation device 200 configured to perform the procedure described above. As shown, the seed implantation device 200 includes a seed 202 and a needle 208. The seed 202 is configured to emit a signal (e.g., a radio frequency (RF) signal, a radioactive signal, a magnetic signal, a direct current signal, an alternating current signal, etc.). The needle 208 is used to place the seed 202 within the target tissue 210 of the breast 212 (or other body part). Thereafter, the needle 208 is removed, which leaves the seed 202 in or near the target tissue 210 of the breast 212. A probe 230 is configured to detect the signal emitted by the seed 202. A computing device 232 (e.g., personal computer, smart phone, tablet, dedicated device, etc.) coupled to the probe 230 is configured to display on a display screen 234 information identifying the location of the target tissue 210 based on the signal received. Thus, the seed 202 and probe 230 enable a surgeon to quickly locate and remove the target tissue 210 and to enter the breast from any location.

In many cases, there is less risk that the seed 202 will migrate within the patient's body between the time of placement and the time of the surgical procedure compared to when the wire is used for localization. However, the seed 202 is relatively expensive, calls for a large bore needle 208 for placement, and eliminates the tactile feedback a surgeon receives when pulling on the wire (e.g., the wire 102 embedded in the target tissue 110 in FIGS. 1A-1B) during the surgical procedure. Thus, the use of the seed 202 for localization also has drawbacks. Accordingly, an improved apparatus for localization of lesions or other tissues in advance of and/or during surgical, diagnostic, or other medical procedures would be useful.

Disclosed herein is a localization apparatus configured to solve one or more of the aforementioned problems with the wire marker device 100 and/or the seed implantation device 200. As will be more fully explained below, the localization apparatus provides tactile feedback to a surgeon, remains securely in place once positioned, and allows the target tissue to be precisely located and effectively removed. In addition, the localization apparatus is inexpensive and easy to manufacture.

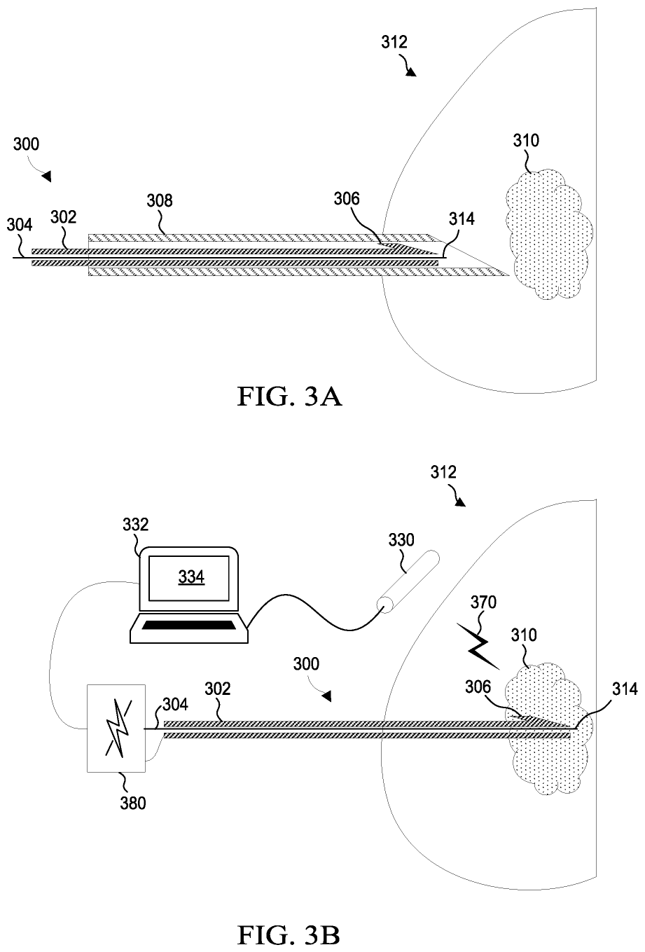

FIGS. 3A and 3B illustrate a localization apparatus 300 according to an embodiment of the present disclosure. The localization apparatus 300 comprises a hollow localization wire 302 and an internal wire 304. In an embodiment, the localization apparatus 300 is approximately twenty-five centimeters (25 cm) in length. In practical applications, a longer or shorter length is possible.

In an embodiment, the hollow localization wire 302 is a tube or catheter-like structure with a hollow cylindrical interior. The hollow localization wire 302 may be constructed from stainless steel, surgical steel, carbon steel, titanium, aluminum, or other material suitable for use during surgery. In an embodiment, the hollow localization wire 302 includes a securing feature 306. The securing feature 306 is configured to secure the hollow localization wire 302 in place relative to target tissue 310. As shown, the securing feature 306 may be disposed proximate the distal end of the hollow localization wire 302. In an embodiment, the securing feature 306 comprises one or more hooks, barbs, spurs, spikes, spines, catches, or some combination thereof.

The internal wire 304 is disposed within the interior of the hollow localization wire 302. In such a configuration, the internal wire 304 is not movable relative to the hollow localization wire 302. In an embodiment, a distal end 314 (a.k.a., a tip) of the internal wire 304 protrudes from the hollow localization wire 302. In an embodiment, the internal wire 304 and the hollow localization wire 302 are generally co-axial with each other.

In an embodiment, the internal wire 304 is configured to be inserted through the hollow interior of the hollow localization wire 302. When force is applied, the internal wire 304 is able to move within, and relative to, the hollow localization wire 302. In an embodiment, the internal wire 304 is inserted through the hollow localization wire 302 until a distal end 314 (a.k.a., a tip) of the internal wire 304 protrudes from the hollow localization wire 302. The internal wire 304 may be constructed from stainless steel, surgical steel, carbon steel, titanium, aluminum, or other material suitable for use during surgery.

As shown in FIG. 3A, an insertion needle 308 may be used to place the localization apparatus 300 within or relative to the target tissue 310 within the breast 312. In an embodiment, the insertion needle 308 is a 18-20 gauge needle. In an embodiment, the insertion needle 308 is smaller than an 18-gauge needle. As shown, the internal wire 304 is forced through the hollow localization wire 302 until the distal end 314 of the internal wire 304 is embedded in the target tissue 310. Thereafter, the insertion needle 308 is removed, which leaves the localization apparatus 300 embedded in or near the target tissue 310 of the breast 312 as shown in FIG. 3B. In an embodiment, approximately 5-10 cm of the localization apparatus 300 is embedded in the breast 312. In an embodiment, no more than approximately 15 cm of the localization apparatus 300 is embedded in the breast 312.

The distal end 314 of the internal wire 304 is configured to emit a signal 370 that permits the target tissue 310 to be located within the breast 312. In an embodiment, the signal 370 has sufficient strength to propagate through the tissue of the breast 312 and out of the body of the patient. By way of example, the signal may be a radio frequency (RF) signal, a magnetic signal, a light signal, an audio signal (e.g., sonar/ultrasound), a direct current voltage, an alternating current voltage, or a combination thereof.

In an embodiment, a probe 330 disposed in proximity to the breast 312 is configured to detect the signal 370 emitted by the distal end 314 of the internal wire 304. In order to detect the signal 370, the probe 330 may be equipped with one or more antennas, sensors, or receivers capable of detecting an RF signal, a magnetic signal, a light signal, an audio signal, a voltage, or some combination thereof. A computing device 332 (e.g., personal computer, smart phone, tablet, etc.) coupled to the probe 330 is configured to display on a display screen 334 information identifying the location of the target tissue 310 based on the signal 370 received.

In an embodiment, the distal end 314 transmits a signal 370 of known strength. The signal 370 is received by the probe 330 and the computing device 332 compares the strength of the received signal to the strength of the signal transmitted. The difference between the strength of the transmitted and received signals may be used to determine a location of the distal end 314 of the internal wire 304 which, in turn, identifies the location of the target tissue 310.

In an embodiment, the distal end 314 transmits a series of signals 370 using a predetermined timing interval. Each received signal 370 is compared to each transmitted signal 370. The time to acquire the received signals 370, any phase differences in the transmitted and received signals 370, and/or any wavelength differences in the transmitted and received signals 370 may be used to determine the location of the distal end 314 of the internal wire 304 which, in turn, identifies the location of the target tissue 310.

In an embodiment, the probe 330 and the computing device 332 may be configured as a single unit. In an embodiment, the single unit (or the probe 330 alone) is sized to be handheld by the surgeon performing the procedure. The probe 330, the computing device 322, or both may be battery operated, rechargeable, or coupled to a wall outlet to obtain power. In an embodiment, the probe 330 and the computing device 332 are coupled via disposable wires or leads and are configured to be easily cleaned (i.e., with or without the need for sterilization). In an embodiment, the probe 330 and/or the computing device 332 is configured to be covered by a disposable sleeve that may be discarded after each surgical procedure.

In an embodiment, energy supplied by the probe 330 is used to induce the distal end 314 of the internal wire 304 to emit the signal 370. For example, when the probe 330 is placed in proximity to the distal end 314, the distal end 314 of the internal wire 304 is energized and emits the signal 370. In an embodiment, a portion of the internal wire 304 opposite the distal end 314 is coupled to the computing device 332. In such a configuration, the computing device 332 may be used to generate signal 370 at the distal end 314 of the internal wire 304.

In an embodiment, a portion of the internal wire 304 and/or a portion of the hollow localization wire 302 opposite the distal end 314 may be coupled to signal generator 380. In such a configuration, the signal generator 380 may be used to generate signal 370 at the distal end 314 of the internal wire 304. In an embodiment, the signal generator 380 causes a current to flow on the internal wire 304 to generate the signal 370. In an embodiment, the current is generated on the internal wire 304 via a direct connection of the internal wire 304 to the signal generator 380. In an embodiment, the current is induced on the internal wire 304 by generating induction between the internal wire 304 and the hollow localization wire 302 to generate the signal 370 (e.g., the signal generator 380 causes an alternating current to flow through a loop formed in the hollow localization wire 302, which produces a changing magnetic field that causes electromagnetic induction to flow through a loop formed in the distal end 314 of the internal wire 304, which produces the signal 370). In an embodiment, the signal generator 380 is an ultrasonic transducer.

In an embodiment, the hollow localization wire 302 includes shielding to ensure that only the distal end 314 of the internal wire 304 protruding from the hollow localization wire 302 is able to transmit the signal 370. That is, the hollow localization wire 302 may be constructed such that any signal generated by portions of the internal wire 304 not protruding from the hollow localization wire 302 is attenuated.

From the foregoing, it should be recognized that the signal 370 and the hollow localization wire 302 collectively enable a surgeon to quickly locate and remove the target tissue 310. In doing so, the signal 370 and the hollow localization wire 302 provide one or more of the benefits of the wire marker device 100 of FIGS. 1A-1B and the seed implantation device 200 of FIGS. 2A-2B without suffering from the drawbacks of those individual devices.

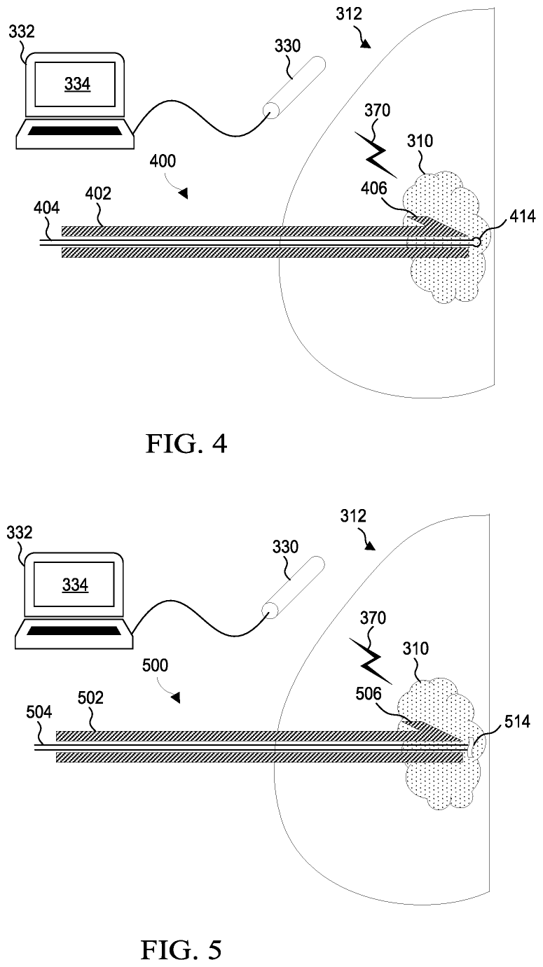

FIG. 4 illustrates a localization apparatus 400 according to an embodiment of the present disclosure. The localization apparatus 400 is similar to the localization apparatus 300 of FIG. 3. That is, the localization apparatus 400 comprises a hollow localization wire 402, which has a securing feature 406, and an internal wire 404. However, the internal wire 404 includes two lengths of wire within the hollow localization wire 402. In addition, the distal end 414 of the internal wire 404 in FIG. 4 forms a loop or contact surface. As shown, the loop or contact surface of the distal end 414 extends beyond the hollow localization wire 402 and is disposed with the target tissue 310. In an embodiment, one or both lengths of the internal wire 404 and/or the hollow localization wire 402 may be coupled to signal generator 380.

As before, a probe 330 disposed in proximity to the breast 312 is configured to detect the signal 370 emitted by the distal end 414 of the internal wire 404. In order to detect the signal 370, the probe 330 may be equipped with one or more antennas, sensors, or receivers capable of detecting an RF signal, a magnetic signal, a light signal, an audio signal, a direct current voltage, an alternating current voltage, or some combination thereof. A computing device 332 coupled to the probe 330 is configured to display on a display screen 334 information identifying the location of the target tissue 310 based on the signal 370 received or detected (e.g., based on the RF signal detected, etc.).

FIG. 5 illustrates a localization apparatus 500 according to an embodiment of the present disclosure. The localization apparatus 500 is similar to the localization apparatus 300, 400 of FIGS. 3-4. That is, the localization apparatus 500 comprises a hollow localization wire 502, which has a securing feature 506, and an internal wire 504. However, the internal wire 504 includes two lengths within the hollow localization wire 502. In addition, the distal end 514 of the internal wire 504 in FIG. 5 forms a magnet or a magnetic choke. In an embodiment, the magnet is formed by coiling a portion of the internal wire 504 around a metal object and introducing a current. As shown, the magnet or magnetic choke of the distal end 514 extends beyond the hollow localization wire 502 and is disposed with the target tissue 310. In an embodiment, one or both lengths of the internal wire 504 and/or the hollow localization wire 502 may be coupled to signal generator 380.

As before, a probe 330 disposed in proximity to the breast 312 is configured to detect the signal 370 emitted by the distal end 514 of the internal wire 504. In order to detect the signal 370, the probe 330 may be equipped with a magnetic sensor (e.g., a Hall effect sensor) capable of detecting, for example, a magnetic signal or some property thereof. A computing device 332 coupled to the probe 330 is configured to display on a display screen 334 information identifying the location of the target tissue 310 based on the signal 370 received or detected (e.g., based on the magnetic field detected, etc.).

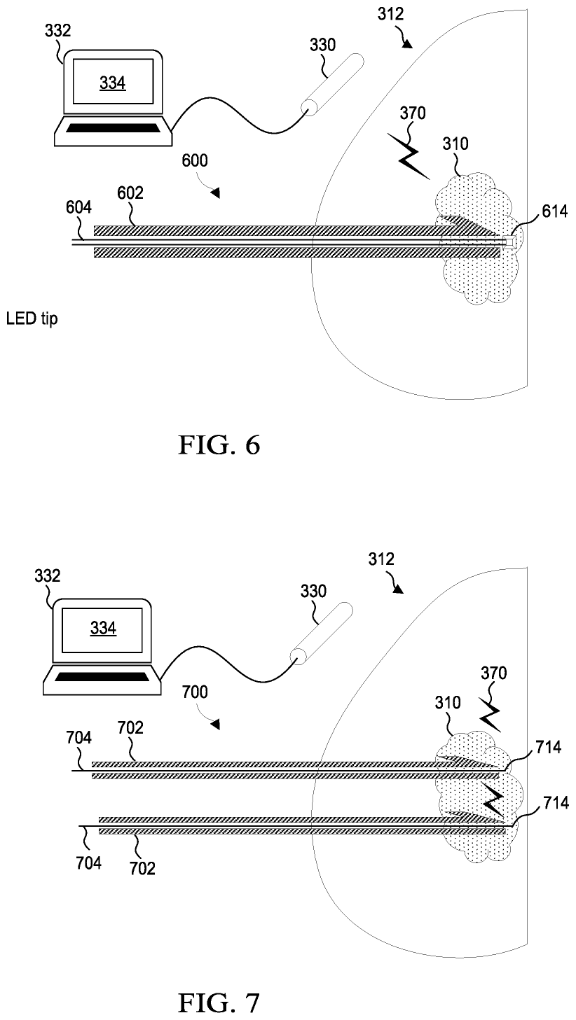

FIG. 6 illustrates a localization apparatus 600 according to an embodiment of the present disclosure. The localization apparatus 600 is similar to the localization apparatus 300, 400, 500 of FIGS. 3-5. That is, the localization apparatus 600 comprises a hollow localization wire 602, which has a securing feature 606, and an internal wire 604. However, the internal wire 604 includes two lengths within the hollow localization wire 602. In addition, the distal end 614 of the internal wire 604 in FIG. 6 comprises a light-emitting feature such as, for example, one or more light emitting diodes (LEDs) or a fiber optic tip. As shown, the light-emitting feature of the distal end 614 extends beyond the hollow localization wire 602 and is disposed with the target tissue 310. In an embodiment, one or both lengths of the internal wire 604 and/or the hollow localization wire 602 may be coupled to signal generator 380.

As before, a probe 330 disposed in proximity to the breast 312 is configured to detect the signal 370 emitted by the distal end 614 of the internal wire 604. In order to detect the signal 370, the probe 330 may be equipped with a photo receiver capable of detecting, for example, light or some property thereof. A computing device 332 coupled to the probe 330 is configured to display on a display screen 334 information identifying the location of the target tissue 310 based on the signal 370 received or detected (e.g., based on the intensity of the light detected, etc.).

FIG. 7 illustrates a localization apparatus 700 according to an embodiment of the present disclosure. The localization apparatus 700 is similar to the localization apparatus 300, 400, 500, 600 of FIGS. 3-6. However, the localization apparatus 700 comprises two hollow localization wires 702, each with a securing feature 706, and two internal wires 704, each with a distal end 714. Each distal end 714 may be similar to one of the distal ends 314, 414, 514, and 614 described above. As shown, each distal end 714 extends beyond the hollow localization wire 702, is disposed with the target tissue 310, and is configured to emit a signal 370. In an embodiment, the two signals 370 are compared to locate the target tissue 310. For example, a difference in the signal strength of the two signals 370 may be used to locate the target tissue 310 or provide a distance to the target tissue 310.

As before, a probe 330 disposed in proximity to the breast 312 is configured to detect the signals 370 emitted by the distal ends 714 of the internal wires 704. In order to detect the signals 370, the probe 330 may be equipped as described above. A computing device 332 coupled to the probe 330 is configured to display on a display screen 334 information identifying the location of the target tissue 310 based on the signals 370 received or detected.

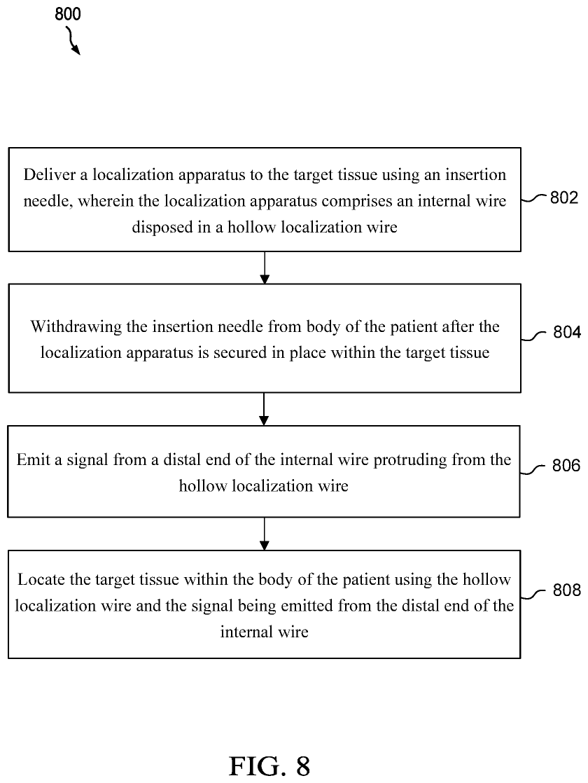

FIG. 8 is a method 800 of locating target tissue (e.g., target tissue 310) in a body of a patient according to an embodiment of the present disclosure. In some circumstances, the target tissue may be disposed in the breast of the patient. The method 800 may be performed by a surgeon in order to localize the target tissue prior to, or during, a surgical procedure to remove or inspect the target tissue.

In block 802, a localization apparatus (e.g., localization apparatus 300-700) is delivered to the target tissue using an insertion needle (e.g., insertion needle 308). The localization apparatus comprises an internal wire (e.g., internal wire 304) disposed in a hollow localization wire (e.g., hollow localization wire 302). In an embodiment, the localization apparatus comprises an internal wire inserted through a hollow localization wire. In block 804, the insertion needle is withdrawn from body of the patient after the localization apparatus is secured in place within the target tissue (e.g., target tissue 310). In an embodiment, the localization apparatus is secured in place by the securing feature (e.g., securing feature 306) of the hollow localization wire.

In block 806, a signal (e.g., signal 370) is emitted from a distal end (e.g., distal end 314) of the internal wire protruding from the hollow localization wire. As described above, the signal may an RF signal, a radioactive signal, a magnetic signal, a direct current signal, an alternating current signal, an audio signal, a visual signal, a plurality of such signals, or a combination of such signals. In block 808, the target tissue is located within the body of the patient and/or removed using the hollow localization wire and the signal being emitted from the distal end of the internal wire.

In an embodiment, the method 800 further comprises inserting the internal wire through the hollow localization wire until the distal end of the internal wire protrudes from the hollow localization wire and is disposed within the target tissue. In an embodiment, the method 800 further comprises inducing the distal end of the internal wire to transmit the signal using a probe, a computing device, or a signal generator.

In an embodiment, the method 800 further comprises displaying on a display screen of the computing device information identifying a location of the target tissue based on the signal received or detected. In an embodiment, the method 800 further comprises measuring a field strength, a signal timing, a signal frequency, or a signal wavelength of the signal to locate the target tissue. In an embodiment, the method 800 further comprises measuring an inductance, a capacitance, a resistance, or some other property generated by, or resulting from, the signal to locate the target tissue.

In an embodiment, the method 800 further comprises providing the hollow localization wire with shielding to ensure that only the distal end of the internal wire protruding from the hollow localization wire is able to transmit the signal.

In an embodiment, the method 800 further comprises delivering a second localization apparatus to the target tissue using a second insertion needle, where the second localization apparatus comprises a second internal wire inserted through a second hollow localization wire. Thereafter, the second insertion needle is withdrawn from the body of the patient after the second localization apparatus is secured in place within the target tissue. Next, a second signal is transmitted from a second distal end of the second internal wire protruding from the second hollow localization wire. Then, the target tissue is located within the body of the patient using the signal being transmitted from the distal end of the internal wire and the second signal being transmitted from the second distal end of the second internal wire. In this embodiment, each signal 370 may have a different characteristic (e.g., frequency) that may be collectively or separately evaluated received by the probe 330 and/or evaluated by the computing device 332.

In an embodiment, the localization apparatus (e.g., localization apparatus 300-700) may be a component of a localization system or a kit. In an embodiment, such a localization system or kit may comprise a localization apparatus, an insertion needle (e.g., insertion needle 308), and a probe 330. In an embodiment, the localization system or a kit may also include a computing device (e.g., computing device 332) and/or a signal generator (e.g., signal generator 380). In an embodiment, the localization system or kit described above may be packaged and sold as a single unit.

While several embodiments have been provided in the present disclosure, it may be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, components, techniques, or methods without departing from the scope of the present disclosure. Other examples of changes, substitutions, and alterations are ascertainable by one skilled in the art and may be made without departing from the spirit and scope disclosed herein.

1. A localization apparatus, comprising:
a hollow localization wire with a securing feature configured to secure the hollow localization wire in place relative to target tissue, wherein the securing feature is integral with the hollow localization wire; and
an internal wire movably disposed in the hollow localization wire, wherein a distal end of the internal wire protruding from the hollow localization wire is configured to emit a signal that was received by a proximal end of the internal wire and that permits the target tissue to be located when a strength of the signal emitted at the distal end of the internal wire protruding from the hollow localization wire is compared to a strength of the signal is detected at a probe.

2. The localization apparatus of claim 1, wherein the signal and the hollow localization wire are both configured to permit the target tissue to be located.

3. The localization apparatus of claim 1, wherein the hollow localization wire includes shielding to ensure only the distal end of the internal wire protruding from the hollow localization wire is able to emit the signal.

4. The localization apparatus of claim 1, wherein the localization apparatus is configured to be received within an insertion needle.

5. The localization apparatus of claim 1, wherein the securing feature comprises a hook or a barb.

6. The localization apparatus of claim 1, wherein the signal is a radio frequency (RF) signal, a magnetic signal, a light signal, an audio signal, a direct current voltage, or an alternating current voltage.

7. The localization apparatus of claim 1, wherein the distal end of the internal wire forms a loop or coil.

8. The localization apparatus of claim 1, wherein the distal end of the internal wire is configured to emit the signal when energized by the probe, when the internal wire is coupled to a signal generator, or when the internal wire is coupled to a computing device.

9.-17. (canceled)

18. A localization system, comprising:
a localization apparatus comprising:
a hollow localization wire with a securing feature configured to secure the hollow localization wire in place relative to target tissue, wherein the securing feature is integral with the hollow localization wire; and
an internal wire configured to be movably disposed in the hollow localization wire, wherein a distal end of the internal wire is configured to emit a signal that was received by a proximal end of the internal wire;
an insertion needle configured to deliver the localization apparatus to the target tissue;
a probe configured to detect the signal emitted by the distal end of the internal wire; and
a computing device configured to compare a strength of the signal emitted at the distal end of the internal wire protruding from the hollow localization wire to a strength of the signal detected at the probe to locate the target tissue.

19. The localization system of claim 18, wherein a signal generator is configured to couple with the internal wire and generate the signal, and wherein the computing device is configured to display on a display screen information identifying a location of the target tissue.

20. The localization system of claim 18, further comprising a signal generator configured to couple to the internal wire, wherein the signal generator is configured to induce the internal wire to generate the signal.

21. The localization apparatus of claim 1, wherein the signal is one of a series of signals, and wherein the distal end of the internal wire is configured to emit the series of signals using a predetermined timing interval.

22. The localization system of claim 18, wherein the signal is one of a series of signals, and wherein the distal end of the internal wire is configured to emit the series of signals using a predetermined timing interval.

23. The localization apparatus of claim 1, wherein the securing feature and the hollow localization wire comprise a single piece of material.

24. The localization system of claim 18, wherein the securing feature and the hollow localization wire comprise one unitary piece of material.
